(19) United States
(12) Patent Application Publication
(10) Pub. No.: US 2008/0140115 A1
(43) Pub. Date: Jun. 12, 2008

(54) Stapling Support Structures

(76) Inventor: Joshua B. Stopek, Yalesville, CT (US)

Correspondence Address: Tyco Healthcare group, 195 Mcdermott Road, North Haven, CT, 06473, US

(21) Appl. No.: 11/660,576

(22) PCT Filed: Aug. 16, 2005
(86) PCT No.: PCT/US05/29280

§ 371 Date: Feb. 16, 2007

Related U.S. Application Data
(60) Provisional application No. 60/602,199, filed on Aug. 17, 2004.

(51) Int. Cl. A61B 17/064; A61B 17/115; B29C 71/00; B29C 71/04

(52) U.S. Cl. 606/219; 227/176.1; 264/347; 264/488

(57) ABSTRACT

The present disclosure relates to support structures for surgical staplers comprising at least on hydrophilic polymer and methods for preparing such structures.

### CROSS-REFERENCE TO RELATED APPLICATION

The present application claims the benefit of and priority to U.S. Provisional Application Ser. No. 60/602,199, filed on Aug. 17, 2004, the entire disclosure of which is incorporated herein by reference.

### BACKGROUND

1. Technical Field

The present disclosure relates to support structures for use in conjunction with a surgical stapling instrument.

2. Background of Related Art

Staples have traditionally been used to replace suturing when joining or anastomosing various body structures, such as, for example, the bowel or bronchus. The surgical stapling devices employed to apply these staples are generally designed to simultaneously cut and seal an extended segment of tissue in a patient, thus, vastly reducing the time and risks of such procedures.

Linear surgical stapling devices are employed by surgeons to sequentially or simultaneously apply one or more linear rows of surgical fasteners, e.g., staples or two-part fasteners, to body tissue for the purpose of joining segments of body tissue together. Such devices generally include a pair of jaws or finger-like structures between which body tissue to be joined is placed. When the stapling device is actuated and/or “fired” firing bars move longitudinally and contact staple drive members in one of the jaws, surgical staples are pushed through the body tissue and into/against an anvil in the opposite jaw thereby crimping the staples closed. If tissue is to be removed, a knife blade can be provided to cut between the rows/lines of staples. Examples of such instruments are described in U.S. Pat. Nos. 4,354,628, 5,014,899 and 5,040,715, the entirety of each of which is incorporated herein by reference.

Annular surgical staplers, for example, an end-to-end anastomosis stapler such as a Model “EEA” instrument are commercially available from United States Surgical, a Division of Tyco Health-Care Group, LP, Norwalk, Conn. and disclosed in U.S. Pat. No. 5,392,979 to Green et al. In general, an end-to-end anastomosis stapler typically places an array of staples into the approximated sections of a patient's bowels or other tubular organs. The resulting anastomosis contains an inverted section of bowel which contains numerous “B” shaped staples to maintain a secure connection between the approximated sections of bowel.

For most procedures, the use of bare staples, with the staples in direct contact with the patient's tissue, is generally acceptable. The integrity of the tissue will normally serve to prevent the staples from tearing out of the tissue and compromising the sealing before healing has occurred. However, in some surgical operations, surgical supports, e.g., meshes, are employed by surgeons to bridge, repair and/or reinforce tissue defects with a patient, especially those occurring in the abdominal wall, chest wall, diaphragm and other musculo-aponeurotic areas of the body. Examples of surgical supports are disclosed in U.S. Pat. Nos. 3,054,406, 3,124,136, 4,347,847, 4,655,221, 4,838,884, 5,002,551, 6,503,257 and WO 03/105698A2, the entire disclosures of which are incorporated herein by this reference.

When the staples are applied in surgical operation utilizing surgical supports (i.e., reinforcing material), the legs of the staple typically pass from the cartridge jaw through a layer of reinforcing material, then through the patient's tissue before encountering the anvil jaw. In an alternative procedure, the legs of the staple typically pass from the cartridge jaw through a first layer of reinforcing material, then through the patient's tissue, and finally through a second layer of reinforcing material before encountering the anvil jaw. With the staples in place, the stapled tissue is clamped between the layers of reinforcing material. The surgical supports described above can be used in conjunction with linear surgical staplers or with annular surgical staplers.

### SUMMARY

The present application is directed in part to support structures configured and adapted for use in conjunction with surgical stapling instruments. The support structures are made from hydrophilic polymers, such as (poly)-hydroxyethylmethacrylate. In certain embodiments, the support structures are prepared by filling a mold with a monomer capable of forming a hydrophilic polymer and at least partially polymerizing the composition within the mold.

The support structures can be configured as an annular ring which is attachable and/or connectable to the distal-most surface of the staple cartridge assembly of an annular stapler. In other embodiments, the support structure be configured as a strip which is attachable and/or connectable to the distal-most surface of the staple cartridge assembly of a linear stapler.

### BRIEF DESCRIPTION OF THE DRAWINGS

By way of example only, preferred embodiments of the disclosure will be described with reference to the accompanying drawings, in which:

FIG. 1 is a top plan view of a support structure in accordance with the present disclosure;

FIG. 1A is a cross-sectional side elevational view of the support structure of FIG. 1, taken along line A-A;

FIG. 1B is a cross-sectional side elevational view of an alternative embodiment of the support structure of FIG. 1, as would be seen along line A-A;

FIG. 1C is a cross-sectional side elevational view of an alternative embodiment of the support structure of FIG. 1, as would be seen along line A-A;

FIGS. 2-4 show various views of a linear stapler equipped with a support structure in accordance with one embodiment of the present disclosure.

FIG. 5 is a top plan view of an alternative embodiment of a support structure in accordance with the present disclosure;

FIG. 5A is a cross-sectional side elevational view of the support structure shown in FIG. 5, taken along line A-A;

FIG. 5B is a cross-sectional side elevational view of an alternative embodiment of the support structure of FIG. 5, as would be seen along line A-A;

FIG. 5C is a cross-sectional side elevational view of an alternative embodiment of the support structure of FIG. 5, as would be seen along line A-A;

FIG. 5D is a cross-sectional side elevational view of an alternative embodiment of the support structure of FIG. 5, as would be seen along line A-A;

FIG. 6 is an enlarged perspective view, with portions broken away, of a distal end of an annular circular stapling apparatus illustrating the placement of a support structure, in accordance with the present disclosure, between the anvil and the staple cartridge of the stapling apparatus;

### DETAILED DESCRIPTION OF PREFERRED EMBODIMENTS

The present support structures for surgical staplers are made from a hydrophilic biomaterial. Examples of suitable hydrophilic biomaterials include polymers formed from one or more of the following monomers: methacrylic acid, acrylic acid, n-vinyl pyrrolidone, potassium sulfopropylacrylate, potassium sulfopropylmethacrylate, acrylamide, dimethylacrylamide, 2-methacryloyloxyethyl phosphorylcholine, hydroxyethylmethacrylate or similar biocompatible water-soluble vinyl monomers. In a particularly useful embodiment, the support structure is formed of (poly)hydroxyethylmethacrylate.

The support structures are prepared using techniques within the purview of those skilled in the art. For example, the support structures can be formed by filling a mold with a composition containing the monomer(s) and, if desired or necessary, initiator, crosslinker, plasticizer and/or biological agent, and polymerizing the composition within the mold. The choice of particular initiators, crosslinkers, etc. will be determined by the specific choice of monomer(s).

Support structures made of poly-(hydroxyethyl methaerylate) (PHEMA) can be synthesized using ⁶⁰Co gamma radiation, UV radiation, or conventional chemical initiated (AIBN, BPO, redox, etc.) free radical polymerization. In a typical preparation method, a composition containing HEMA monomer, AlBN as an initiator and diethyleneglycol dimethacrylate (DEGDMA) as a crosslinker is poured into a glass mold and polymerized at approximately 65° C. for 1.5 hours. Resulting support structures are washed repeatedly with water and dried in vacuo. In another preparation method, PHEMA support structures can be prepared using radiation polymerization (600 mC source, 295-1180 rad/min, 0.05-1 Mrad) without the need of chemical initiator or crosslinker, and using the same washing/drying regiment. In yet other embodiments, polymerization can also be conducted using aqueous monomer solutions of various concentration to afford buttress materials of varied mechanical and physical properties (films tailored for different tissues, staples, procedures, etc.).

The equilibrium water content (EWC), swelling, and mechanical properties of the PHEMA support structures are controlled by crosslink density (radiation conditions or DEOGMA concentration). The thickness of the support structure is controlled by the volume of the monomer composition polymerized in the mold. Suitable thickness for the support structures is in the range of about 0.1 to about 5 mm.

The support structure can be any shape, and will normally be configured to correspond to and cover at least a portion of a staple line applied by a surgical stapler. Suitable shapes include rectangular strips (e.g., for linear staplers) and annular rings (e.g., for annular staplers). The cross-sectional shape of the support structure have any cross-sectional profile, such as, for example, generally rectangular, circular, ovoid, triangular, arcuate, etc.

The present support structures can also be surface modified following film formation. For example, a PHEMA support structure can be surface modified with polymeric phospholipids for improved hemocompatibility and tissue interaction using gamma radiation grafting.

In another embodiment, the surface of the surface of the support structures can be patterned or templated in the nano-meso-micro scale to accommodate preferential tissue interaction at the tissue/buttress interface. Such architecture or patterns can prevent or minimize post-operative tissue adhesions and superfluous collagen deposition, but afford desired mechanical and biophysical support for wound healing.

The composition from which the support structure is made may also contain one or more medically and/or surgically useful substances such as drugs, enzymes, growth factors, peptides, proteins, dyes, diagnostic agents or hemostasis agents or any other pharmaceutical used in the prevention of stenosis. Non-limiting examples of suitable medically and/or surgically useful substances include: antimicrobials, antibiotics, anti-fungals, anti-virals, monoclonal antibodies, polyclonal antibodies, antimicrobial proteins/peptides (whole and fragments), enzymes, gene therapy, viral particles, chemotherapeutics, anti-inflammatories, NSAIDS, steroids, telomerase inhibitors, growth factors (TGF family, interleukin superfamily, fibroblast derived GFs, macrophage derived GFs, etc.), extracellular matrix molecules (laminin, thrombospondin, collagen, fibronectin, synthetic ECM, etc.), cell adhesion molecules, polysaccharides (hyaluronic acid, carboxymethyl cellulose, alginate, sulfonated dextran, heparin sulfate, chitosan, etc.) and others. These agents can be incorporated in situ into the composition used the make the support structure or post loaded onto the polymerized support structure using techniques within the purview of those skilled in the art. For example, the medically and/or surgically useful substances can be freely mixed or loaded, electronically or ionically bound, covalently immobilized, chelated, or encapsulated in particles, micelles, aggregates, or any nano-meso-micro solids of varied dimension, shape morphology and dispersion/suspension ability.

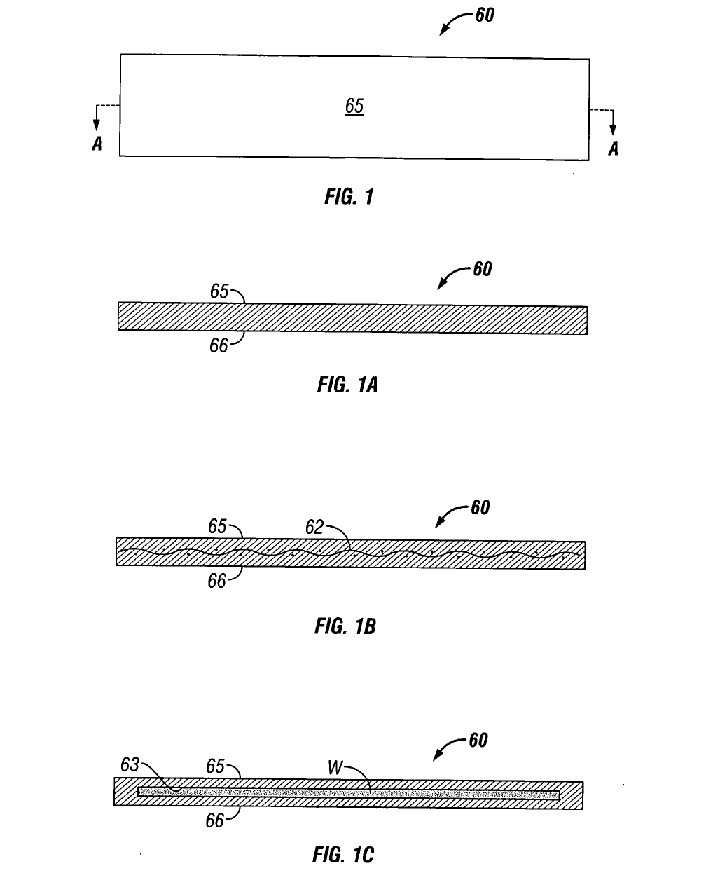

Referring initially in detail to FIG. 1, a support structure 60 in accordance with the present disclosure intended for use with a linear stapler has a generally rectangular shape and is made of a hydrophilic biomaterial. The support strip 60 includes first surface 65 and second surface 66, one of which will be in contact with the tissue being stapled, depending on whether the strip 60 is adhered to the staple cartridge or the anvil of the stapling apparatus. As seen in FIG. 1A, the support structure 60 has a generally rectangular cross-section.

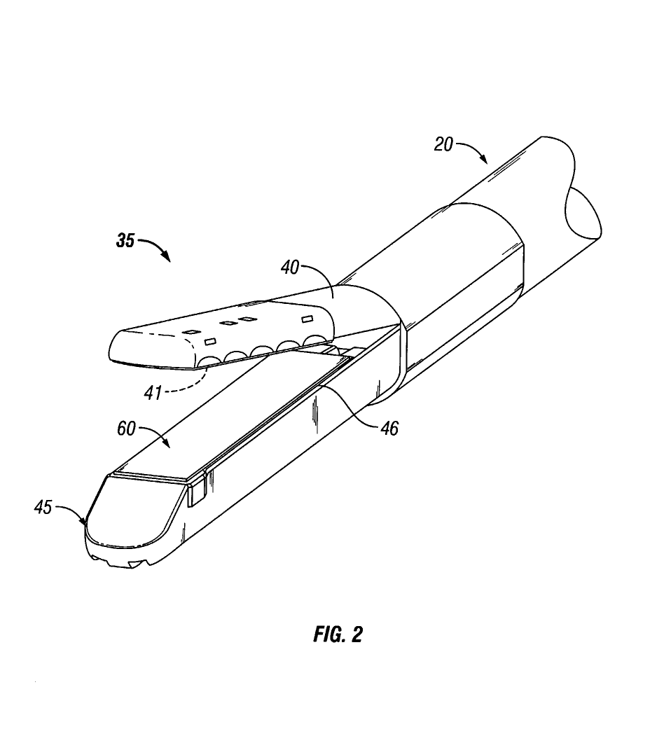

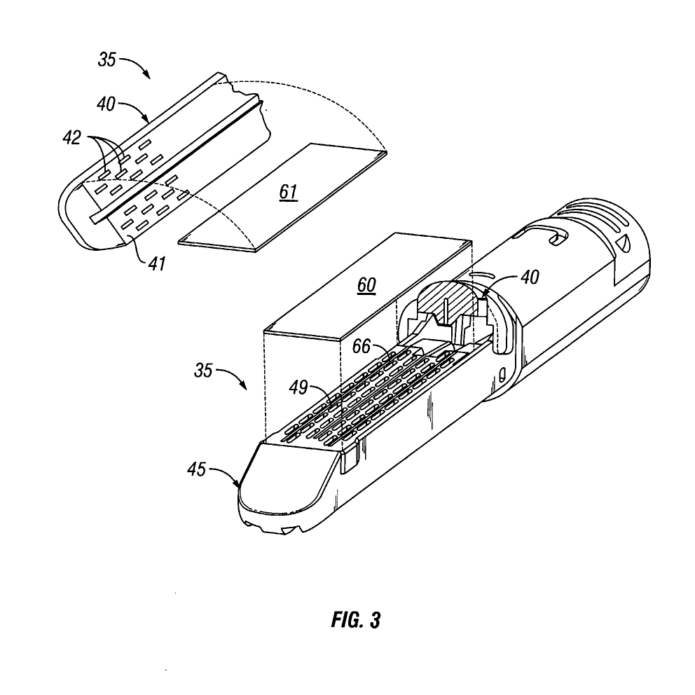

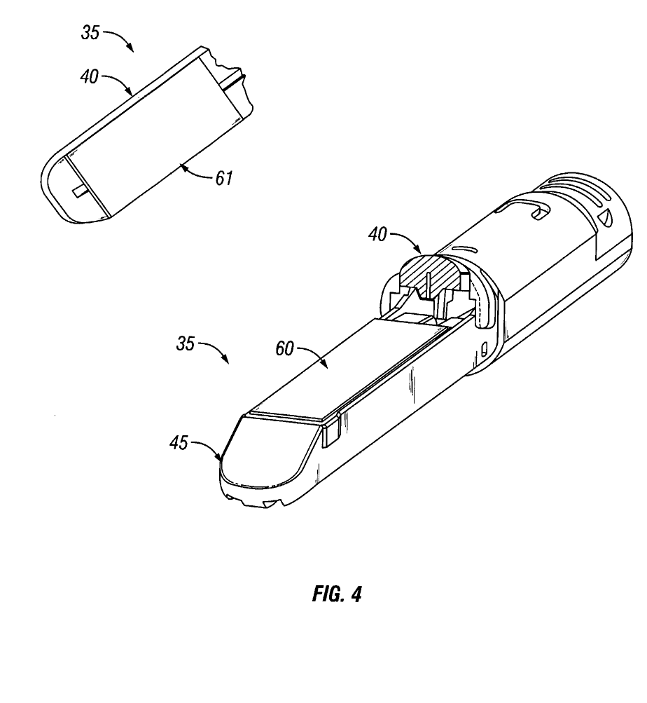

Turning now to FIGS. 2-4, support structures in the form of a strip in accordance with the present disclosure are shown generally as 60, 61. End 35 of surgical stapling device 20 has a first and a second tissue clamping member movable between an open position for receiving tissue therebetween, and a closed position for stapling tissue therebetween. The first tissue clamping member has a removable staple cartridge 45 mounted therein. The second tissue clamping member is a moveable anvil 40, which is opposite to the first tissue clamping member. Staple cartridge 45 contains a plurality of staples 49 housed within. Moveable anvil 40 moves from the open position of FIG. 2 to a closed position adjacent to the removable staple cartridge 45 (not shown). During operation of the stapler, the staples 49 are driven from the removable staple cartridge 45, through the buttress strips 60 and 61, and are formed into tight “B” shapes (not shown) against the anvil 40. The ejection of the staples 49 from the removable staple cartridge 45 also releases the second buttress strip 61 from the second tissue clamping surface 41 and forms the “U” shaped staples 49 into “B” shapes. The “U” shaped staples 49 are formed into “B” shapes by driving them through the second buttress strip 61 attached to the second tissue clamping surface 41 and against the staple pockets 42 within the anvil 40. As the wire of the staple 49 is driven into the staple pocket 42, the ends of the staple wire curl around into the “B” shape, and dislodge the support structure 60 from cartridge 45 and also detaches support structure 61 from the anvil 40. The surgical stapling device 20 and removable staple cartridge 45 are generally well known and described, for example, in U.S. Pat. Nos. 4,354,628, 5,014,899 and 5,040,715.

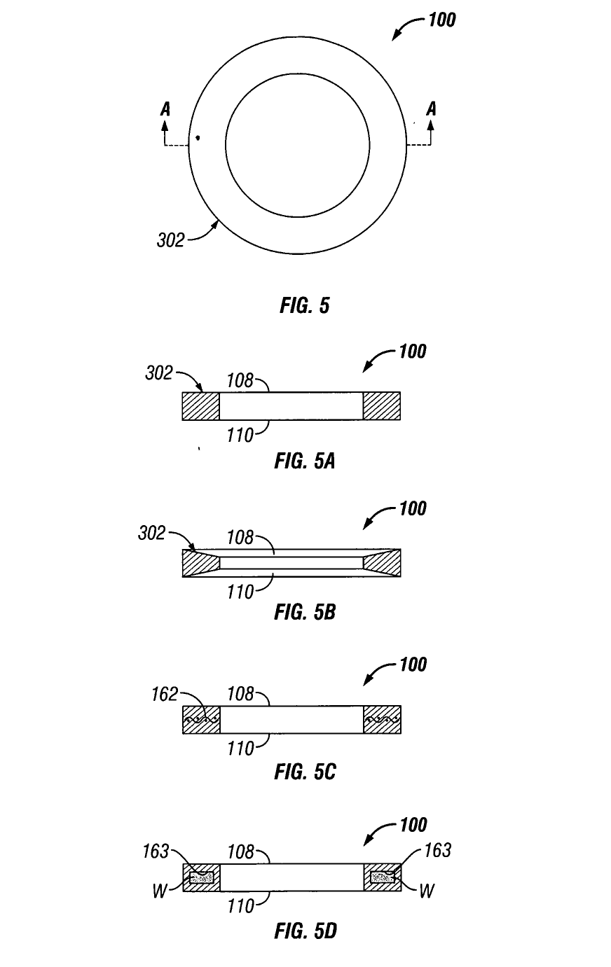

In other embodiments, the support structure 100 made in accordance with this disclosure can have a ring-like structure as shown in FIG. 5 and is intended for use in combination with an annular stapler, such as the type commonly employed for performing anastomoses. In cross section, support structure 100 can have a generally rectangular configuration as seen in FIG. 5A, or may have a tapered cross sectional shape as seen in FIG. 5B.

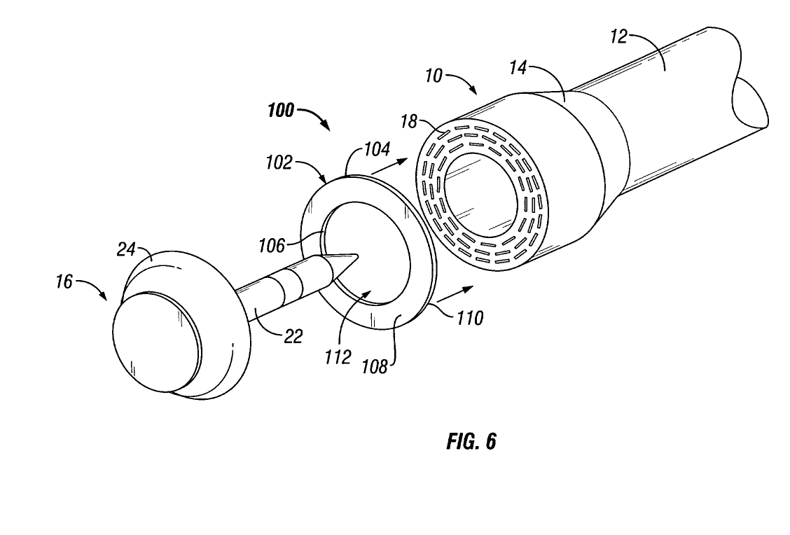

Referring initially in detail to FIG. 6, a surgical stapling support structure in the form of a ring, in accordance with an embodiment of the present disclosure, is generally shown as 100. Ring 100 includes an annular ring 102 defined by an outer terminal edge 104, an inner terminal edge 106, an upper surface 108 and a lower surface 110. Inner terminal edge 106 of ring 100 defines a central opening 112. One of upper surface 108 or lower surface 110 will be in contact with the tissue being stapled, depending on whether the ring 100 is adhered to the staple cartridge or the anvil of the stapling apparatus.

As seen in FIG. 6, ring 100 cooperates with a circular stapling apparatus 10. Stapling apparatus 10 includes an elongated neck 12 having a staple cartridge assembly 14 operatively coupled to an end thereof and an anvil assembly 16 configured and adapted to removably engage the distal end of staple cartridge assembly 14. Staple cartridge assembly 14 is configured and adapted to expel an annular array of staples (not shown) out of the distal end thereof. Preferably, staple cartridge assembly 14 includes a plurality of annular rows of staple slots 18 having staples therein. Anvil assembly 16 includes a shaft 22 which is adapted to be releasably mounted within staple cartridge assembly 14 and an anvil 24 which is mounted on shaft 22 and is oriented to be positioned towards the distal end of staple cartridge assembly 14. Anvil 24 is provided with an annular array of staple forming cups 19, conforming to the number of annular rows and number of staple slots 18, the cups being configured and adapted to form staples, e.g. into a B-shape, as they are expelled from staple cartridge assembly 14.

Ring 100 is releasably attached to either anvil assembly 16 or staple cartridge assembly 14. Alternatively, anvil assembly 16 and staple cartridge assembly 14 can both have a reinforcing ring 100 disposed thereon (not shown) to provide a tissue/support “sandwich” upon actuation and/or firing of stapling apparatus 10.

The attachment of ring 100, to circular stapling apparatus 10 should be secure enough to prevent ring 100 from slipping off of stapling apparatus 10, yet not be so strong as to inhibit separation of reinforcing ring 100 from stapling device 10 after stapling device 10 has been actuated. Such releasable attachment can advantageously be effected by employing a plurality of pins as described in commonly assigned U.S. Pat. No. 5,542,594, the entire contents of which are incorporated herein by reference. It is further contemplated that an adhesive, for example, a releasable adhesive, can be employed to achieve releasable attachment. Alternatively, a plurality of longitudinally spaced clips (not shown herein) may also be employed as the means for securing ring 100 to stapling apparatus 10. The precise number and location of pins and/or clips or the amount or placement of continuity of spots or lines of adhesive is not critical so long as ring 100 is releasably attached to stapling apparatus 10. It should, of course be understood that the hydrophilic polymer chosen to make the support structure advantageously can have a certain degree of adhesive properties, thus avoiding the need for any supplemental attachment means.

It is contemplated that a fibrous reinforcing element, such as a surgical grade mesh, can be incorporated into the support structures in accordance with the present disclosure. For example, in FIG. 1B, strip 60 is shown to include mesh 62 therein, and in FIG. 5C, ring 100 is shown to include mesh 162 therein. Suitable fibrous reinforcing elements can be made from a biocompatible non-absorbable (i.e., permanent) material, such as, for example “TEFLON” which is a registered trademark owned by DuPont de Nemours & Co., or a biocompatible absorbable material. The biocompatible materials can be woven, knit or non-woven. Bio-absorbable materials include those fabricated from homopolymers, copolymers or blends obtained from one or more monomers selected from the group consisting of glycolide, glycolic acid, lactide, lactic acid, p-dioxanone, α-caprolactone and trimethylene carbonate. Non-absorbable materials include those that are fabricated from such polymers as polyethylene, polypropylene, nylon, polyethylene terephthalate, polytetrafluoroethylene, polyvinylidene fluoride, and the like. Further non-absorbable materials include and are not limited to stainless steel, titanium and the like.

In an alternative embodiment of a support structure in accordance with the present disclosure, a reservoir is provided which retains an amount of a biological adhesive or other useful substance therein. For example, as seen in FIG. 1C, strip 60 includes reservoir 63. As another example, as seen in FIG. 5D, ring 100 includes reservoir 163. While a biological adhesive has been disclosed as being retained within the reservoir, it is envisioned that reservoir can retain any type of wound closure material “W” therein. It is envisioned that wound closure material “W” can include one or a combination of adhesives, hemostats, and sealants. Surgical biocompatible wound closure materials which can be retained in the reservoir include adhesives whose function is to attach or hold organs, tissues or structures, sealants to prevent fluid leakage, and hemostats to halt or prevent bleeding. Examples of adhesives which can be employed include protein derived, aldehyde-based adhesive materials, for example, the commercially available albumin/glutaraldehyde materials sold under the trade designation BioGlue™ by Cryolife, Inc., and cyanoacrylate-based materials sold under the trade designations Indermil™ and Derma Bond™ by Tyco Healthcare Group, LP and Ethicon Endosurgery, Inc., respectively. Examples of sealants, which can be employed, include fibrin sealants and collagen-based and synthetic polymer-based tissue sealants. Examples of commercially available sealants are synthetic polyethylene glycol-based, hydrogel materials sold under the trade designation CoSeal™ by Cohesion Technologies and Baxter International, Inc. Examples of hemostat materials, which can be employed, include fibrin-based, collagen-based, oxidized regenerated cellulose-based and gelatin-based topical hemostats. Examples of commercially available hemostat materials are fibrinogen-thrombin combination materials under sold the trade designations CoStasis™ by Tyco Healthcare Group, LP, and Tisseel™ sold by Baxter International, Inc. Hemostats herein include astringents, e.g., aluminum sulfate, and coagulants.

While the above disclosure has related generally to two specific types of stapling apparatus, it should be understood that the support structures according to the present disclosure can be utilized in connection with any type of stapling apparatus and the stapling of any type of tissue. Further while the support structure has been disclosed herein in connection with certain embodiments and certain structural and procedural details, it is clear that changes, modifications or equivalents can be used by those skilled in the art. Therefore, the above description should not be construed as limiting, but merely as exemplifications of preferred embodiments. Those skilled in the art will envision other modifications within the scope and spirit of the present disclosure.

What is claimed is:

1. A medical device comprising:
a support structure made from at least one hydrophilic polymer, the support structure being adapted and configured for use in connection with a surgical stapler.

2. A medical device as in claim 1 wherein the at least one hydrophilic polymer comprises at least one monomer selected from the group consisting of methacrylic acid, acrylic acid, n-vinyl pyrrolidone, potassium sulfopropylacrylate, potassium sulfopropylmethacrylate, acrylamide, dimethylacrylamide, 2-methacryloyloxyethyl phosphorylcholine, hydroxyethylmethacrylate, polyhydroxyethylmethacrylate, biocompatible water-soluble vinyl monomers and combinations thereof.

3. A medical device as in claim 1 wherein the at least one hydrophilic polymer is poly-hydroxyethylmethacrylate.

4. A medical device as in claim 1 wherein the support structure further comprises at least one additional ingredient selected from the group consisting of initiators, crosslinkers, plasticizers, biological agents and combinations thereof.

5. A medical device as in claim 1 wherein the support structure has a thickness ranging from about 0.1 to about 5 mm.

6. A medical device as in claim 1 wherein the support structure has a generally rectangular shape.

7. A medical device as in claim 1 wherein the support structure has a generally annular shape.

8. A medical device as in claim 1 wherein the support structure further comprises polymeric phospholipids on a surface of the support structure.

9. A medical device as in claim 1 wherein the support structure further comprises at least one medically useful substance.

10. A medical device as in claim 1 wherein the support structure further comprises at least one medically useful substance is selected from the group consisting of drugs, enzymes, growth factors, peptides, proteins, dyes, diagnostic agents, hemostasis agents, and combinations thereof.

11. A medical device as in claim 1 wherein the support structure further comprises an adhesive coating.

12. A medical device as in claim 1 wherein the support structure further comprises a fibrous reinforcing element.

13. The support structure of claim 12 wherein the fibrous reinforcing element comprises a woven, knit, or non-woven structure.

14. A medical device as in claim 1 wherein the support structure further comprises a reservoir containing a substance selected from the group consisting of biological adhesives, sealants, hemostats, wound closure materials and combinations thereof.

15. A method for preparing a support structure for surgical staplers comprising:
introducing a composition comprising at least one hydrophilic polymer in a mold;
at least partially polymerizing the composition in the mold; and
removing the support structure form the mold.

16. A method as in claim 15 wherein the step of at least partially polymerizing the composition comprises using gamma radiation, UV radiation, or conventional chemical initiated free radical polymerization.

17. A method as in claim 15 wherein a composition comprising at least one monomer selected from the group consisting of methacrylic acid, acrylic acid, n-vinyl pyrrolidone, potassium sulfopropylacrylate, potassium sulfopropylmethacrylate, acrylamide, dimethylacrylamide, 2-methacryloyloxyethyl phosphorylcholine, hydroxyethylmethacrylate, poly-hydroxyethylmethacrylate, biocompatible water-soluble vinyl monomers and combinations thereof is introduced into a mold.

18. A method as in claim 15 wherein a composition comprising polyhydroxyethylmethacrylate is introduced into a mold.

19. A method as in claim 15 wherein a composition comprising at least one an additional ingredient selected from the group consisting of initiators, a crosslinkers, plasticizers, biological agents and combinations thereof is introduced into a mold.

20. A method as in claim 15 wherein a composition comprising at least one medically useful substance is introduced into a mold.

21. A method as in claim 20 wherein the at least one medically useful substance is selected from the group consisting of drugs, enzymes, growth factors, peptides, proteins, dyes, diagnostic agents, hemostasis agents, and combinations thereof.

22. A method as in claim 15 further comprising introducing a fibrous reinforcing element into the mold.

23. A method as in claim 15 wherein the composition is introduced into a generally rectangular mold.

24. A method as in claim 15 wherein the composition is introduced into a generally annular mold.

25. A method comprising
positioning a support structure made from at least one hydrophilic polymer adjacent a tissue contacting surface of a surgical stapler; and
firing the surgical stapler to drive staples through the support structure and tissue, whereby the support structure is secured to the tissue by the staples.

26. A method as in claim 25 wherein the support structure is positioned adjacent an anvil of a surgical stapler.

27. A method as in claim 25 wherein the support structure is positioned adjacent a staple cartridge of a surgical stapler.

28. A method as in claim 25 wherein the at least one hydrophilic polymer comprises at least one monomer selected from the group consisting of methacrylic acid, acrylic acid, n-vinyl pyrrolidone, potassium sulfopropylacrylate, potassium sulfopropylmethacrylate, acrylamide, dimethylacrylamide, 2-methacryloyloxyethyl phosphorylcholine, hydroxyethylmethacrylate, poly-hydroxyethylmethacrylate, biocompatible water-soluble vinyl monomers and combinations thereof.

29. A method as in claim 25 wherein the at least one hydrophilic polymer comprises hydroxyethylmethacrylate.

30. A method as in claim 25 wherein the support structure further comprises a fibrous reinforcement element.

31. A method as in claim 25 wherein the support structure further comprises a reservoir containing a substance selected from the group consisting of biological adhesives, sealants, hemostats, wound closure materials and combinations thereof.
